(19) United States
(12) Patent Application Publication
(10) Pub. No.: US 2025/0041275 A1
(43) Pub. Date: Feb. 6, 2025

(54) COMPOSITION FOR USE IN THE TREATMENT OF A DISEASE

(71) Applicants: RAMBAM MED-TECH LTD., Haifa (IL); TECHNION RESEARCH & DEVELOPMENT FOUNDATION LIMITED, Haifa (IL)

(72) Inventors: Iyad KHAMAYSI, Haifa (IL); Salim HADAD, Haifa (IL); Zaid ABASSI, Haifa (IL)

(73) Assignees: RAMBAM MED-TECH LTD., Haifa (IL); TECHNION RESEARCH & DEVELOPMENT FOUNDATION LIMITED, Haifa (IL)

(21) Appl. No.: 18/911,431

(22) Filed: Oct. 10, 2024

Related U.S. Application Data
(63) Continuation-in-part of application No. PCT/IL2023/050433, filed on Apr. 27, 2023.
(60) Provisional application No. 63/335,978, filed on Apr. 28, 2022.
(60) Provisional application No. 63/567,970, filed on Mar. 21, 2024.

(51) Int. Cl. A61K 31/405; A61K 31/194; A61K 31/196; A61K 47/54; A61P 13/12

(52) U.S. Cl. CPC A61K 31/405; A61K 31/194; A61K 31/196; A61K 47/549; A61P 13/12

(57) ABSTRACT

The invention relates generally to the field of compositions including one or more conjugates of di-sugar and a nonsteroidal anti-inflammatory drug (NSAID), and use of same in methods for treating a disease or a disorder in a subject in need thereof.

### CROSS-REFERENCE TO RELATED APPLICATIONS

This application is a Continuation-In-Part of PCT Application No. PCT/IL2023/050433, filed Apr. 27, 2023, which claims the benefit of priority of U.S. Provisional Patent Application No. 63/335,978 filed Apr. 28, 2022. This application also claims the benefit of priority of U.S. Provisional Patent Application No. 63/567,970 filed Mar. 21, 2024. The contents of which are all incorporate herein by reference in their entirety.

### FIELD OF THE INVENTION

The invention relates generally to the field of compositions comprising one or more trehalose conjugates and is directed to methods of using the same such as for treating a disease or a disorder in a subject.

### BACKGROUND

Acute pancreatitis (AP) is a common disease in gastroenterology with an increasing global incidence, where it accounts for about 2% of all hospitalized patients. AP is a complex inflammatory syndrome that results from many etiologies where gallstones, alcohol and ERCP are the leading causes. About 20% of patients who experienced a first AP-attack will develop recurrent attacks (RAP) and approximately one third of the latter continue to end-stage chronic pancreatitis (CP). Despite the great advances in medicine, the worldwide mortality rate among AP patients remained high, imposing an important burden on the health care system. It is widely accepted that excessive stimulation of the pancreas or direct destructive insults obstruct the outflow of zymogen granules, where they are proteolytically activated in the acinar cells by lysosomal enzymes mainly cathepsin B and eventually causing acute cell injury. This adverse reaction is further exacerbated by neutrophilic enzymes and transcription factors, which lead to the production of various proinflammatory cytokines, including tumor necrosis factor (TNF)-α, interleukin (IL)-1, IL-6, and IL-8, along conversion of trypsinogen into trypsin—a phenomenon also referred to as auto-digestion. Moreover, the proinflammatory stimuli upregulates cyclooxygenase (COX) 2, a key enzyme responsible for the generation of prostaglandins, leukotrienes, and thromboxanes.

In light of the vague characterization of the mechanistic pathways responsible for AP, the treatment options targeting a specific underlying cause remain elusive and the current therapy relies mainly on pain killers and hydration, where opioids being the most frequently prescribed analgesics for pain relief of patients with AP. As AP is secondary to pancreatic parenchymal inflammation, non-steroidal anti-inflammatory drugs (NSAIDs) are also often used. Previous studies have highlighted the keen involvement of heparanase in the pathogenesis of inflammatory diseases including AP. Specifically, it has been evidenced that pancreatic heparanase expression and activity are significantly increased following cerulein-induced AP. Moreover, pancreas edema and inflammation, as well as the induction of cytokines and signaling molecules following cerulein administration were attenuated markedly by PG545 and SST0001, selective heparanase inhibitors, implying that heparanase plays a significant role in AP. Notably, all the above features appear even more pronounced in transgenic mice over expressing heparanase, suggesting that these mice can be utilized as a sensitive model system to reveal the molecular mechanism by which heparanase functions in AP. Therefore, there is an ongoing need to develop novel medications for specific and efficient treatment of AP.

Inflammatory brain disease, also referred to as inflammatory disease of the central nervous system (CNS), is a condition where the brain and/or spinal cord become inflamed. Inflammation in the brain causes irritation and swelling of brain tissue or blood vessels and can lead to brain damage over the long term.

Chronic brain inflammation leads to measurable brain shrinkage, especially in the areas associated with Alzheimer's disease, the 6th leading cause of death. Chronic brain inflammation leads to mental fatigue, brain fog, and memory loss. Chronic brain inflammation has been linked to numerous neurological and psychiatric disorders, including depression, anxiety, substance abuse, schizophrenia, bipolar disorder, Alzheimer's, and Parkinson's.

Encephalitis is a rare form of acute brain inflammation that is usually caused by a viral or bacterial infection. Symptoms include fever, headache, seizures, stiff neck and back, and mental confusion. Encephalitis can cause brain damage and even death.

Non-Steroidal Anti-inflammatory Drug (NSAID) are extensively used for the treatment of inflammation and pain. NSAIDs act by inhibiting Cyclooxygenase (COX)-1 and/or COX-2 activity, thereby reducing intracellular synthesis of prostaglandins, being associated with inflammation and/or pain. However, most NSAIDs are not able to cross the blood brain barrier (BBB) in an amount sufficient for inducing a therapeutic effect. Therefore, there is an ongoing need to develop methods for enhancing BBB penetration of known NSAIDs, e.g., for use in the treatment of brain inflammation and for reducing COX activity within the brain.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to the first aspect, there is provided a pharmaceutical composition, comprising a therapeutically effective amount of a conjugate and a pharmaceutically acceptable carrier, wherein the conjugate comprises a di-sugar covalently bound to a Non-Steroidal Anti-inflammatory Drug (NSAID).

According to another aspect, there is provided a conjugate, comprising trehalose covalently bound to a Non-Steroidal Anti-inflammatory Drug (NSAID) selected from the group comprising diclofenac, naproxen, diflunisal, salsalate, ibuprofen, indomethacin, mefenamic acid, meclofenamic acid, clonixin, licofelone or a combination thereof.

According to another aspect, there is provided a pharmaceutical composition comprising the conjugate of the invention, and a pharmaceutically acceptable carrier.

According to another aspect, there is provided a method for preventing or treating a COX-related disease, a Hep-related disease, or both, in a subject in need thereof, the method comprising administering to the subject a therapeutically effective amount of any one of: (a) the pharmaceutical composition of the invention; and (b) the conjugate of the invention, thereby, preventing or treating a COX-related disease, a Hep-related disease, or both, in the subject.

According to another aspect, there is provided a method for preventing or treating a kidney disease in a subject in need thereof, the method comprising administering to the subject a therapeutically effective amount of any one of: (a) the pharmaceutical composition of the invention; and (b) the conjugate of the invention, thereby, thereby, preventing or treating a kidney disease in the subject.

According to another aspect, there is provided a method for inhibiting or reducing enzymatic activity within a subject in need thereof, comprising administering the pharmaceutical composition of the invention to the subject.

In some embodiments, the covalently bound is via an ester bond between a carboxy group of the NSAID and a hydroxy group of the di-sugar.

In some embodiments, the NSAID comprises one or more compounds selected from the group consisting of: aspirin, diclofenac, indomethacin, naproxen, diflunisal, salsalate, ibuprofen, mefenamic acid, meclofenamic acid, clonixin, licofelone, a COX-2 inhibitor including any pharmaceutically acceptable salt, pharmaceutically active derivative, and a combination thereof.

In some embodiments, the di-sugar is trehalose.

In some embodiments, the NSAID is selected from selected from the group consisting of: aspirin, diclofenac, indomethacin, and any combination thereof.

In some embodiments, the pharmaceutical composition is for use in the prevention or treatment of a disease or disorder in a subject in need thereof.

In some embodiments, the disease or disorder is selected from the group consisting of: inflammation, pain, and both.

In some embodiments, the disease or disorder is or comprises pancreatitis.

In some embodiments, the disease or disorder is associated with cyclooxygenase (COX) activity, Heparanase (Hep) activity, or both.

In some embodiments, the disease or disorder is a kidney disease.

In some embodiments, the kidney disease is acute kidney injury (AKI).

In some embodiments, the trehalose is covalently bound to a carboxy group of the NSAID.

In some embodiments, covalently bound is via a bond selected from an ester, an amide, a thioester, a carbamate, a carbonate ester, a carbamide, a thiocarbamate, a phosphonate, a phosphodiester, a sulfonate ester, or any combination thereof.

In some embodiments, the trehalose is covalently bound via a hydroxy group at position 2, at position 6 or both.

In some embodiments, the conjugate is represented by Formula 1:

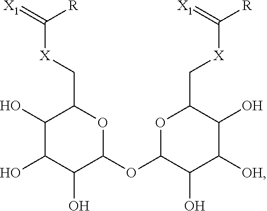

or of Formula 1a:

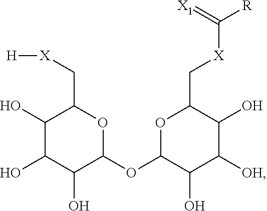

wherein each R independently comprises decarboxylated diclofenac, decarboxylated indomethacin, decarboxylated naproxen, decarboxylated diflunisal, decarboxylated salsalate, decarboxylated paracetamol, decarboxylated ibuprofen, decarboxylated mefenamic acid, decarboxylated meclofenamic acid, decarboxylated clonixin, or decarboxylated licofelone, and each X or X₁ independently comprises O, S, or NH.

In some embodiments, the conjugate comprises any of:

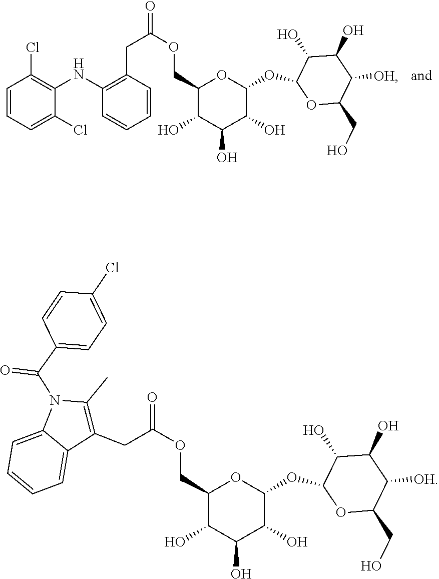 [SMILES: O=C(Cc1ccccc1Nc1c(Cl)cccc1Cl)OC[C@H]1O[C@H](O[C@H]2O[C@H](CO)[C@@H](O)[C@H](O)[C@H]2O)[C@H](O)[C@@H](O)[C@@H]1O ; Cc1c(CC(=O)OC[C@H]2O[C@H](O[C@H]3O[C@H](CO)[C@@H](O)[C@H](O)[C@H]3O)[C@H](O)[C@@H](O)[C@@H]2O)c2ccccc2n1C(=O)c1ccc(Cl)cc1]

In some embodiments, the treating or preventing comprises reducing any one of: kidney/body weight (% g), creatinine level, blood urea nitrogen level, and any combination thereof, in the subject compared to a control subject.

In some embodiments, the creatinine level is blood creatinine level, urine creatinine level, or both.

In some embodiments, the preventing or treating comprises reducing expression level of a marker selected from neutrophil gelatinase-associated lipocalin (NGAL), kidney injury molecule-1 (KIM-1), or both, in a kidney of the subject, compared to a control subject.

In some embodiments, the expression level comprises transcript level of the marker, protein level of the marker, or both.

In some embodiments, the control subject is afflicted with the kidney disease and is not being treated with any one of the pharmaceutical composition of the invention and the conjugate of the invention.

In some embodiments, reducing enzymatic activity comprises preventing or treating any condition selected from the group consisting of: inflammation, fever, and pain, in the subject.

In some embodiments, inflammation comprises pancreatitis.

### BRIEF DESCRIPTION OF THE FIGURES

FIGS. 1A-1C include bar graphs showing effects of either PG545, SST0001, Aspirin or combined therapy on cerulein-induced pancreatitis as evident by serum levels of lipase (1A), amylase (1B) and pancreatic index (Pancreas/Body weight ratio) (1C) in WT mice and Hpa-Tg animals. *, p<0.05, **, p<0.01***, p<0.001 compared to saline group; #, p<0.05 ##, p<0.01, ###, p<0.001 compared to cerulein group; $, p<0.05, $$, p<0.01, $$$, p<0.001 compared to combination group.

FIGS. 2A-2E and 2A′-2G′ include micrographs showing histopathology images of WT and Hpa-Tg mice injected with either saline or cerulein in the presence or absence of PG545, SST0001, Asp or combined pretreatment. Pancreas tissues were collected 24 h thereafter, and 5 micron sections from formalin-fixed, paraffin-embedded samples were stained for H&E. Shown are representative photomicrographs at X20 original magnification. FIGS. 2A-2E present Hematoxylin-stained samples of WT and Hpa-Tg mice injected with either saline (2A) or cerulein (2B) in the presence or absence of Asp (2C), PG545 (2D) or combined pretreatment PG545+Asp (2E). FIGS. 2A′-2G′ present Eosin-stained samples of WT and Hpa-Tg mice injected with either saline (2A′) or cerulein (2B′) in the presence or absence of PG545 (2C′), SST0001 (2D′), Asp (2E′) or combined pretreatment PG545+Asp (2F′) and PG545+Asp+ SST0001 (2G′).

FIGS. 3A-3D include bar graphs and micrographs showing the effect of pre-treatment with Aspirlose (mono or Diester) in cerulein-induced pancreatitis as evident by serum levels of lipase (3A), amylase (3B) and pancreatic index (Pancreas/Body weight ratio) (3C) and histological alterations (3D) in WT mice and Hpa-Tg animals. *, p<0.05, ** p<0.01 compared to saline group; #, p<0.05, ##, p<0.01 compared to cerulein group; $, p<0.05 compared to combination group.

FIGS. 4A-4C include bar graphs showing the effect of post-treatment with Aspirlose (mono or Diester) in cerulein-induced pancreatitis as evident by serum levels of lipase (4A), amylase (4B) and pancreatic index (Pancreas/Body weight ratio) (4C) in Hpa-Tg animals. *, p<0.05 compared to saline group; #, p<0.05 compared to cerulein group; $, p<0.05 compared to combination group.

FIGS. 5A-5D include bar graphs and micrographs showing the effect of pre-treatment with Indose (conjugate of Indomethacin and Trehalose) or Diclose (conjugate of Diclofenac and Trehalose) (16 or 32 mg/kg i.p., respectively) in cerulein-induced pancreatitis as evident by serum levels of lipase (5A), amylase (5B) pancreatic index (Pancreas/Body weight ratio (P/M)) (5C) and histological analysis (5D) in WT animals. *, p<0.05 compared to saline group; #, p<0.05 compared to cerulein group; $, p<0.05 compared to combination group.

FIGS. 6A-6C include bar graphs and micrographs showing the effect of pre-treatment with Indose (conjugate of Indomethacin and Trehalose) or Diclose (conjugate of Diclofenac and Trehalose) (16 or 32 mg/kg i.p., respectively) in cerulein-induced pancreatitis as evident by serum levels of lipase (6A), amylase (6B) and pancreatic index (Pancreas/Body weight ratio (P/M)) (6C) in Hpa-Tg animals. *, p<0.05 compared to saline group; #, p<0.05 compared to cerulein group; $, p<0.05 compared to combination group.

FIGS. 7A-7C include bar graphs showing the effect of pre-treatment with Indose (conjugate of Indomethacin and Trehalose) or Diclose (conjugate of Diclofenac and Trehalose) (16 mg/kg i.p.) on the severity of acute kidney injury (AKI) in Hpa-Tg mice. (7A) Kidney/weight (% g); (7B) Creatinine levels (mg/dL); and (7C) Blood Urea Nitrogen levels (mg/dL). * P<0.05, Sham vs. AKI; #P<0.05, AKI before vs. after treatment.

FIGS. 8A-8C include bar graphs showing the comparative effect of pre-treatment with Indose (conjugate of Indomethacin and Trehalose) or Diclose (conjugate of Diclofenac and Trehalose) (16 mg/kg i.p.) on the severity of acute kidney injury (AKI) in male vs. female Hpa-Tg mice. (7A) Kidney/weight (% g); (7B) Creatinine levels (mg/dL); and (7C) Blood Urea Nitrogen levels (mg/dL). * P<0.05, Sham vs. AKI; #P<0.05, AKI before vs. after treatment.

FIG. 9 includes histological micrographs showing the effect of pre-treatment with Indose (conjugate of Indomethacin and Trehalose) or Diclose (conjugate of Diclofenac and Trehalose) (16 mg/kg i.p.) on histological adverse alterations characterizing acute kidney injury (AKI) in male vs. female Hpa-Tg mice. F, female; M, male.

FIGS. 10A-10F include photographs showing the effect of pre-treatment with Indose (10B and 10E) or Diclose (10C and 10F) (16 mg/kg i.p.) on gross morphology characterizing acute kidney injury (AKI) in male (10A-10C) vs. female (10C-10F) Hpa-Tg mice. Control AKI (10A and 10D).

FIGS. 11A-11B include bar graphs showing the effect of pre-treatment with Indose (Ind) or Diclose (Dic) (16 mg/kg, ip) on the expression of biomarkers of acute kidney injury (AKI) in male vs. female Hpa-Tg mice. Neutrophil gelatinase-associated lipocalin (NGAL; 11A); Kidney Injury Molecule-1 (KIM-1; 11B).

FIGS. 12A-12C include bar graphs showing the comparative effect of pre-treatment with Indose (conjugate of Indomethacin and Trehalose) or Diclose (conjugate of Diclofenac and Trehalose) (16 mg/kg or 32 mg/kg, i.p., respectively) on the severity of acute kidney injury (AKI) in male vs. female wild-type (WT) mice (FVB/N strain). (12A) Kidney/Body weight (% g); (12B) Creatinine levels (mg %); and (12C) Blood Urea Nitrogen levels (mg %). *<0.05—Sham vs. AKI; #P<0.05—AKI before vs. after treatment; and $—Male vs. female.

FIGS. 13A-13C include bar graphs showing the comparative effect of post-treatment with Indose (conjugate of Indomethacin and Trehalose) or Diclose (conjugate of Diclofenac and Trehalose) (16 mg/kg or 32 mg/kg, i.p., respectively) on the severity of acute kidney injury (AKI) in male vs. female wild-type (WT) mice (FVB/N strain). (13A) Kidney/Body weight (% g); (13B) Creatinine levels (mg %); and (13C) Blood Urea Nitrogen levels (mg %). * P<0.05—Sham vs. AKI; #P<0.05 AKI before vs. after treatment; and $—Male vs. female.

### DETAILED DESCRIPTION

In one aspect of the invention disclosed herein, there is a conjugate, comprising a disaccharide covalently bound to a compound selected from the group comprising diclofenac, naproxen, diflunisal, salsalate, paracetamol, ibuprofen, indomethacin, mefenamic acid, meclofenamic acid, clonixin, and licofelone or any combination thereof.

In some embodiments, non-limiting examples of the disaccharide are: Sucrose, Lactose, Maltose, Trehalose, Cellobiose, Chitobiose, Kojibiose, Nigerose, Isomaltose, β,β-Trehalose, α,β-Trehalose, Sophorose, Laminaribiose, Gentiobiose, Turanose, Trehalulose, Maltulose, Leucrose, Isomaltulose, Gentiobiulose, Mannobiose, Melibiose, Allolactose, Melibiulose, Lactulose, Rutinose, Rutinulose, Xylobiose, or any combination thereof.

In some embodiments, the disaccharide comprises two monosaccharide units connected to each other via a glycosidic bond, wherein each of the monosaccharide units is independently selected from a hexose and a pentose. In some embodiments, each of the monosaccharide units is a hexose. In some embodiments, the disaccharide is trehalose or a derivative thereof (e.g., a conformer, a diastereomer, an enantiomer).

In some embodiments, the conjugate comprises trehalose covalently bound to a compound selected from the group comprising diclofenac, naproxen, diflunisal, salsalate, ibuprofen, paracetamol, indomethacin, mefenamic acid, meclofenamic acid, clonixin, and licofelone or any combination thereof. In some embodiments, the compound is or comprises at least one NSAID selected from diclofenac, naproxen, ibuprofen, indomethacin, and paracetamol, or any combination thereof. In some embodiments, the conjugate comprises trehalose covalently bound to a plurality of compounds, wherein the compound is as described hereinabove. In some embodiments, the compound is devoid of aspirin.

In some embodiments, trehalose is covalently bound via a hydrolysable bond. In some embodiments, trehalose is covalently bound via a biodegradable or biocleavable bond. Such biodegradable or biocleavable bonds are well-known in the art and comprise inter alia an amide bond, an ester bond, and a disulfide bond. In some embodiments, the covalent bond is selected from an ester, an amide, a thioester, a carbamate, a carbonate ester, a carbamide, a thiocarbamate, a phosphonate, a phosphodiester, a sulfonate ester, or any combination thereof. In some embodiments, the covalent bond is selected from an ester, an amide, a thioester, or any combination thereof.

In some embodiments, the conjugate comprises trehalose covalently bound to one or more compounds via an ester bond.

In some embodiments, the trehalose is covalently bound to the compound via at least one hydroxyl group of trehalose. In some embodiments, the trehalose is covalently bound to a carboxy group of the compound. In some embodiments, the conjugate comprises trehalose covalently bound to carboxy group of the compound, wherein covalently bound is via a hydroxyl group of trehalose at position 2, at position 6, or both.

In some embodiments, the conjugate is of Formula 1:

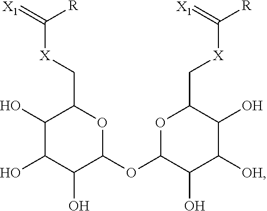

or of Formula 1a:

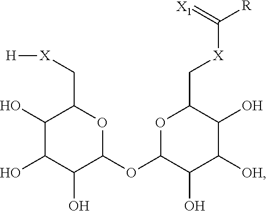

wherein each R independently is or comprises a decarboxylated NSAID selected from decarboxylated diclofenac, decarboxylated naproxen, decarboxylated diflunisal, decarboxylated salsalate, decarboxylated paracetamol, decarboxylated ibuprofen, decarboxylated indomethacin, decarboxylated mefenamic acid, decarboxylated meclofenamic acid, decarboxylated clonixin, or decarboxylated licofelone, including any salt and/or any pharmaceutically active derivative thereof, and each X or X₁ independently comprises O, S, or NH.

In some embodiments, the conjugate is of Formula:

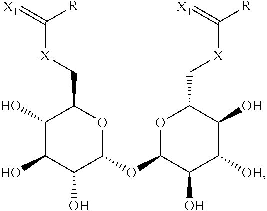

or of Formula:

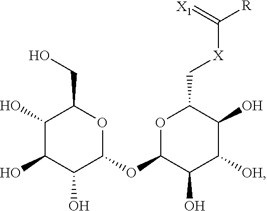

wherein each R, X and X₁ are as disclosed hereinabove. In some embodiments, each X or X₁ is O.

As used herein the term “derivative” refers to a small molecule derived from the abovementioned NSAIDs, and having an NSAID functionality or activity. The NSAID activity can be determined by well-known methods, such as methods suitable for determining Cyclooxygenase inhibition activity. Additionally, the NSAID activity of a conjugate of the invention comprising the NSAID derivative can be evaluated by determining the reduction ability of the enhanced Hep activity (e.g. as evaluated by the reduction of the elevated lipase/amylase concentration).

Furthermore, the term “derivative” encompasses any structurally similar functional derivative of the abovementioned NSAIDs, wherein structurally similar is at least 90%, at least 95%, at least 96%, at least 97%, at least 98%, at least 99% structure similarity, including any range between.

In some embodiments, the term “structure similarity” refers to a fingerprint similarity between two molecules. The term “fingerprint similarity” is well-understood by a skilled artisan. In some embodiments, the fingerprint similarity is calculated based on circular fingerprints, substructure keys-based fingerprints, and/or topological or path-based fingerprints.

Exemplary circular fingerprints include but are not limited to: Molprint 2D, ECFP (or Morgan fingerprint), FCFP, etc. In some embodiments, the term “structure similarity” as used herein, is calculated by Morgan fingerprint.

In some embodiments, the term “decarboxylated NSAID”, comprises an NSAID devoid of a carboxy group, i.e., having a bond instead of the carboxy group. In some embodiments, the bond is an attachment point of R to the —C(═X₁) X group of the conjugate molecule, as represented by Formula 1 or 1a.

In some embodiments, the conjugate is of Formula 2:

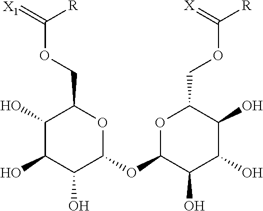

or of Formula 2a:

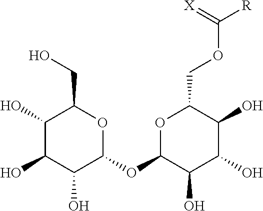

wherein X and R are as described hereinabove.

In some embodiments, each R is independently selected from the group comprising:

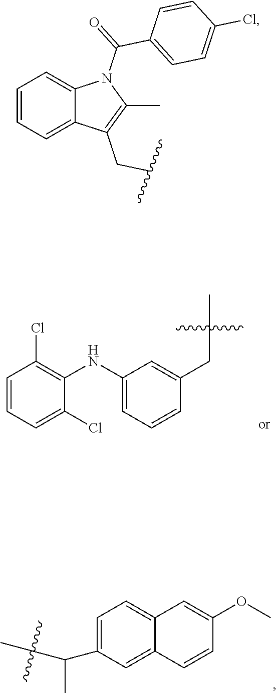 [SMILES: *Cc1c(C)n(C(=O)c2ccc(Cl)cc2)c2ccccc12 ; *Cc1cccc(Nc2c(Cl)cccc2Cl)c1 ; *C(C)c1ccc2cc(OC)ccc2c1]

wherein the wavy bond represents the attachment point of R to the —C(═X₁)X group, or —C(═X)O group of the conjugate molecule.

In some embodiments, the NSAID comprises a compound inhibiting a cyclooxygenase (COX) enzyme activity. In some embodiments, the NSAID comprises a compound selectively inhibiting COX-1 enzyme activity. In some embodiments, the NSAID comprises a compound selectively inhibiting COX-2 enzyme activity. In some embodiments, the NSAID comprises a compound inhibiting COX-1 and COX-2 enzyme activity.

In some embodiments, non-limiting examples of NSAIDs include but are not limited to aspirin, diclofenac, naproxen, indomethacin, diflunisal, salsalate, ibuprofen, mefenamic acid, meclofenamic acid, clonixin, and licofelone, including any pharmaceutically active derivative or a combination thereof.

In some embodiments, the conjugate comprises trehalose covalently bound to at least one of aspirin, diclofenac, indomethacin, and naproxen including any salt or any combination thereof.

In some embodiments, the conjugate of the invention comprises any one of:

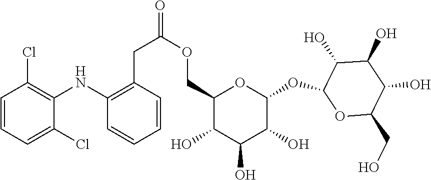 [SMILES: O=C(Cc1ccccc1Nc1c(Cl)cccc1Cl)OC[C@H]1O[C@H](O[C@H]2O[C@H](CO)[C@@H](O)[C@H](O)[C@H]2O)[C@H](O)[C@@H](O)[C@@H]1O]

(also used herein as “Diclose”),

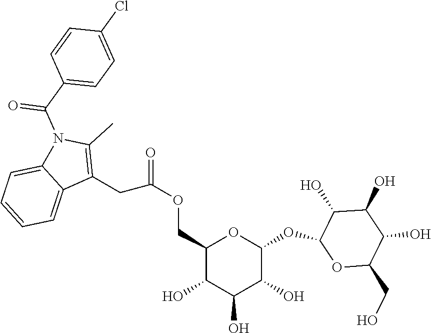 [SMILES: Cc1c(CC(=O)OC[C@H]2O[C@H](O[C@H]3O[C@H](CO)[C@@H](O)[C@H](O)[C@H]3O)[C@H](O)[C@@H](O)[C@@H]2O)c2ccccc2n1C(=O)c1ccc(Cl)cc1]

(also used herein as “Indose”),

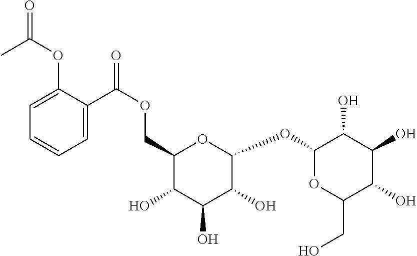 [SMILES: CC(=O)Oc1ccccc1C(=O)OC[C@H]1O[C@H](O[C@H]2OC(CO)[C@@H](O)[C@H](O)[C@H]2O)[C@H](O)[C@@H](O)[C@@H]1O]

(also used herein as “Asprilose-mono ester”), and

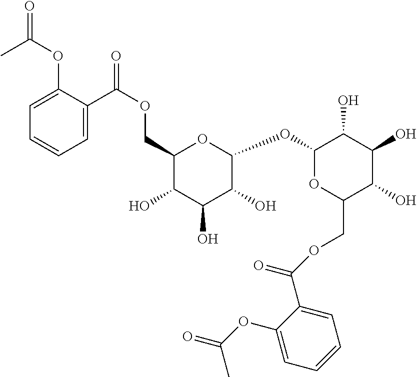 [SMILES: CC(=O)Oc1ccccc1C(=O)OCC1O[C@H](O[C@H]2O[C@H](COC(=O)c3ccccc3OC(C)=O)[C@@H](O)[C@H](O)[C@H]2O)[C@H](O)[C@@H](O)[C@@H]1O]

(also used herein as “Asprilose-di ester”).

### Pharmaceutical Composition (NSAID)

In another aspect of the invention, there is provided a pharmaceutical composition, comprising a therapeutically effective amount of any one of the conjugates of the invention and a pharmaceutically acceptable carrier. In some embodiments, the pharmaceutical composition comprises a therapeutically effective amount of the conjugate of the invention as an active ingredient.

As used herein, the term “therapeutically effective amount” refers to an amount effective, at dosages and for periods of time necessary, to achieve the desired therapeutic or prophylactic result in a subject. The exact dosage form and regimen would be determined by the physician according to the patient's condition.

In some embodiments, the pharmaceutical composition comprises the conjugate of the invention, a pharmaceutically acceptable salt thereof or both. In some embodiments, pharmaceutically acceptable salt comprises the conjugate of the invention and a pharmaceutically acceptable anion.

In some embodiments, examples of pharmaceutically acceptable anions include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises of the conjugate of the invention as the sole therapeutically active ingredient. In some embodiments, the conjugate of the invention is the only therapeutically active ingredient within the pharmaceutical composition of the invention. In some embodiments, the pharmaceutical composition is devoid of additional therapeutically active ingredients.

In some embodiments, the pharmaceutical composition comprises trehalose or a pharmaceutically active derivative thereof, covalently bound to a NSAID. In some embodiments, the pharmaceutical composition comprises trehalose or a pharmaceutically active derivative thereof, covalently bound to an NSAID. In some embodiments, the pharmaceutical composition comprises trehalose or a pharmaceutically active derivative thereof, covalently bound to one or more compounds selected from the group comprising aspirin, diclofenac, naproxen, diflunisal, salsalate, paracetamol, ibuprofen, indomethacin, mefenamic acid, meclofenamic acid, clonixin, and licofelone or any combination thereof. In some embodiments, the pharmaceutical composition comprises trehalose or a pharmaceutically active derivative thereof, covalently bound to one or more compounds selected from the group comprising aspirin, diclofenac, naproxen, diflunisal, salsalate, paracetamol, ibuprofen, indomethacin, mefenamic acid, meclofenamic acid, clonixin, and licofelone, including any salt or any combination thereof.

In some embodiments, the conjugate of the invention present in the pharmaceutical composition is of pharmaceutical grade purity, i.e., is characterized by a chemical purity of at least about 90%, at least about 95%, greater than 95%, or greater than 99%, and between 90 and 99.999%, between 90 and 95%, between 90 and 97%, between 95 and 99%, including any range in between.

### Carrier

In some embodiments, the pharmaceutical composition comprises a therapeutically effective amount of the conjugate of the invention and a pharmaceutically acceptable carrier.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

In some embodiments, the carrier improves: (i) water solubility of the active ingredient, (ii) permeability of the active ingredient through lipid membranes or both.

As used herein, the term “carrier” refers to any component of a pharmaceutical composition (e.g. a material which is in a liquid state, or in a solid state at room temperature) that is not the active agent such as a diluent, an adjuvant, an excipient, or a vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

In some embodiments, the carrier is or comprises any of: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpincol, terpinen-4-ol, thymol, and thymoquinone. One skilled in the art will appreciate, that a particular carrier used within the pharmaceutical composition of the invention may vary depending on the route of administration.

In some embodiments, suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

In some embodiments, non-limiting examples of carriers for pharmaceutical compositions being in the form of a cream include but are not limited to: non-ionic surfactants (e.g., glyceryl monolinoleate, glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the carrier is a liquid carrier. In some embodiments, the carrier is water, or an aqueous solution substantially devoid of an organic solvent. In some embodiments water is used when the active agent is water soluble. In some embodiments, the active agent water solubility is sufficient to be administrated intravenously. As used herein, the term “water soluble” including any grammatical form thereof refers to the ability of the compound undergo a dissolution in water (or in an aqueous solution), so as to form an aqueous solution, substantially devoid of a particulate matter (i.e., undissolved aggregates or solid particles of the compound). The presence of particles/aggregates in the aqueous solution can be determined by various methods, such as DLS.

In some embodiments, the carrier is an aqueous solution. In some embodiments, non-limiting examples of aqueous solutions are: saline solutions, aqueous dextrose buffers such as phosphate buffers and aqueous solutions comprising glycerol.

In some embodiments, the carrier constitutes in total, between 0.1% to 99.99%, between 0.1 and 50%, between 30 and 90%, between 20 and 99.99%, between 90 and 99.99%, between 70 and 90% by weight of the pharmaceutical composition of the invention.

In some embodiments, the pharmaceutical composition further comprises any one of: a wetting agent, emulsifying agent, pH buffering agents (such as acetate, citrate, or phosphate buffer), antibacterial agent (e.g., benzyl alcohol or methyl parabens), antioxidant (such as ascorbic acid or sodium bisulfite), tonicity agent (e.g., sodium chloride or dextrose), or any combination thereof.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions are well known to those of skill in the art and may influence the physical state, solubility, stability, rate of in vivo release, and rate of in vivo clearance.

In some embodiments, liposomes for use with the presently described composition are formed from standard vesicle-forming lipids which generally include one or more neutral phospholipid, and/or positively charged phospholipid, and/or negatively charged phospholipid, optionally a PEG-lipid, and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiments, the pharmaceutical composition is in a form of but not limited to: an emulsion, a liquid solution, a gel, a paste, a suspension, a dispersion, an aerosol, an ointment, a cream, a foam, suppository, a patch, a pill, a capsule, lozenge, wafer, ampoule, vial or pre-filled syringe, pad or gelled stick.

In some embodiments, the pharmaceutical composition is a liquid at a temperature between 15 and 45° C. In some embodiments, the pharmaceutical composition is a solid at a temperature between 15 to 45° C. In some embodiments, the pharmaceutical composition is in a form of a suppository.

In some embodiments, the pharmaceutical composition is a semi-liquid at a temperature between 15 and 45° C. It should be understood that the term “semi-liquid”, is intended to mean materials which are flowable under pressure and/or shear force. In some embodiments, semi-liquid compositions include creams, ointments, gel-like materials, and other similar materials. In some embodiments, the pharmaceutical composition is a semi-liquid composition, characterized by a viscosity in a range from 31,000-800,000 cps.

In some embodiments, the pharmaceutical composition is in the form of a cream. In some embodiments, the cream further comprises a thickener.

In some embodiments, non-limiting examples of thickeners include, but are not limited to microcrystalline cellulose, a starch, a modified starch, gum tragacanth, gelatin, and a polymeric thickener (e.g., polyvinylpyrrolidone) or any combination thereof.

In some embodiments, the pharmaceutical composition of the invention is stable for a time period ranging for at least 10 h, at least 24 h, at least 2 d, at least 10 d, at least 20 d, at least 1 m, at least 6 m, at least 1 year, including any value and range therebetween. As used herein, the term “stable” means that the pharmaceutical composition retains at least 80%, at least 85%, at least 90%, at least 95%, at least 99%, by weight of the initial conjugate loading after the above mentioned time period, including any range between. The term stability as used herein encompasses chemical stability of the composition (i.e. at least 90%, at least 95%, at least 99%, or between 90 and 95%, between 90 and 99% by dry weight of each of constituents of the composition retains its chemical identity, as determined by well-known analytical methods, such as HPLC, GC/LC-MS), as well as physical stability (e.g. physical appearance, mechanical/physical integrity, uniformity, etc.)

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for systemic use.

In some embodiments, the pharmaceutical composition is for use in the prevention and/or treatment of a condition in a subject. In some embodiments, the condition comprises a disease or disorder selected from inflation, pain, or both. In some embodiments, the condition comprises a disease or disorder associated with inflammation and/or pain. In some embodiments, the pharmaceutical composition comprises a disaccharide covalently bound to a Non-Steroidal Anti-inflammatory Drug (NSAID). In some embodiments, the pharmaceutical composition comprises a disaccharide covalently bound to a plurality of NSAIDs, wherein NSAIDs are as described hereinabove.

In some embodiments, the pharmaceutical composition is for use in the increasing internalization of an NSAID into a cell, as compared to a similar pharmaceutical composition comprising an unconjugated NSAID, or as compared to the conjugate of the invention devoid of the pharmaceutically acceptable carrier, i.e., which is not formulated within the pharmaceutical composition (also referred to herein as “control”). In some embodiments, the conjugate of the invention has an increased internalization ability of an NSAID into a cell, as compared to an unconjugated NSAID. In some embodiments, the conjugate of the invention has an increased bioavailability, as compared to an unconjugated NSAID. In some embodiments, the conjugate of the invention has increased specificity or therapeutic efficacy for treatment of a disease or condition disclosed herein, as compared to the control. In some embodiments, the pharmaceutical composition is for increasing any one of: bioavailability, specificity, and/or therapeutic efficacy of an NSAID, as compared to a similar pharmaceutical composition comprising an unconjugated NSAID, or as compared to the conjugate of the invention devoid of the pharmaceutically acceptable carrier, i.e., which is not formulated within the pharmaceutical composition. In some embodiments, the cell is a human cell. In some embodiments, the cell is a pancreatic tissue cell. In some embodiments, the cell is or comprises an endocrine cell. In some embodiments, the cell is or comprises an alpha cell, a beta cell, a delta cell, an epsilon cell, a gamma cell, or any combination thereof.

In some embodiments, the term “increased” and the term “enhanced” are used herein interchangeably and refer to at least 10%, at least 20%, at least 30%, at least 50%, at least 100%, at least 2 times, at least 4 times, at least 5 times, at least 7 times, at least 10 times enhancement, as compared to the control, including any range between.

In some embodiments, the pharmaceutical composition and/or the conjugate of the invention is for use in the prevention or treatment of a disease or a disorder in a subject in need thereof. In some embodiments, the disease or disorder comprises inflammation. In some embodiments, the pharmaceutical composition is for use in the prevention or treatment of a disease or disorder associated with inflammation. In some embodiments, inflammation comprises neuroinflammation. In some embodiments, the pharmaceutical composition is for use in the prevention or treatment of a neuroinflammatory disorder. In some embodiments, inflammation comprises a peripheral inflammatory disease.

In some embodiments, non-limiting examples of peripheral inflammatory diseases include but are not limited to pancreatitis (such as acute pancreatitis, chronic pancreatitis and autoimmune pancreatitis), arthritis, atherosclerosis, asthma, chronic obstructive pulmonary disease (COPD), acute respiratory distress syndrome (ARDS) (e.g. viral and/or bacterial infection related ARDS, toxicity related ARDS, sepsis related ARDS etc.), thromboembolic diseases, chronic dermatitis, chronic hepatitis, cirrhosis, an inflammatory disorder of the intestinal tract, inflammatory bowel disease (IBD), colitis, and Crohn's disease or any combination thereof.

In some embodiments, the pharmaceutical composition is for use in the prevention or treatment of pancreatitis. In some embodiments, the pharmaceutical composition is for use in the prevention or treatment of a disease or disorder associated with pancreatitis. In some embodiments, the pharmaceutical composition is for use in the prevention or treatment of a disease or disorder selected from the group comprising acute pancreatitis, chronic pancreatitis, autoimmune pancreatitis, and post-endoscopic retrograde cholangiopancreatography (ERCP) pancreatitis or any combination thereof.

In some embodiments, the disease or disorder is associated with an abnormal (e.g. enhanced) enzymatic activity in at least one cell, or within a subject, as compared to the activity of the same enzyme(s) within a healthy cell or within a healthy subject. In some embodiments, the abnormal enzymatic activity comprises enhanced cyclooxygenase (COX) activity, enhanced heparinase (Hep) activity, or both. In some embodiments, the conjugate of the invention is capable of inducing an inhibition or reduction of COX activity and/or Hep activity within a cell or within the subject (e.g. within a peripheral organ of the subject). In some embodiments, the abnormal (e.g. enhanced) Hep activity is associated with enhanced lipase and/or amylase levels within a sample derived from the subject.

In some embodiments, the disease or disorder is selected from brain inflammation, abnormal (e.g. increased) blood clotting, pain and fever, or any combination thereof. In some embodiments, the pharmaceutical composition is for use in the prevention or treatment of brain inflammation. In some embodiments, the pharmaceutical composition is for use in the prevention or treatment of a disease or disorder associated with brain inflammation. In some embodiments, the pharmaceutical composition is for use in the prevention or treatment of pain (e.g., headache, migraine etc.). In some embodiments, the pharmaceutical composition is for use in the prevention or treatment of fever. In some embodiments, the pharmaceutical composition is for use in the prevention or treatment of abnormal blood clotting.

In some embodiments, the pharmaceutical composition is for use in inhibition or reduction of an enzymatic activity in a subject in need thereof. In some embodiments, the enzymatic activity comprises cyclooxygenase (COX) activity, heparinase (Hep) activity or both. In some embodiments. In some embodiments, the pharmaceutical composition is for use in inhibition or reduction of COX activity and/or Hep activity within a peripheral organ of the subject.

In some embodiments, the pharmaceutical composition/conjugate of the invention is for use in inhibition or reduction of cyclooxygenase (COX) activity and/or Hep activity within a brain of a subject in need thereof. In some embodiments, reduction of COX activity comprises reduction of the activity of COX-1, COX-2, or both. In some embodiments, the pharmaceutical composition/conjugate is for selective inhibition or reduction of COX-1 or COX-2 activity. In some embodiments, the pharmaceutical composition/conjugate is for selective inhibition or reduction of Hep activity. In some embodiments, selective is at least 70%, at least 75%, at least 80%, at least 85%, at least 90%, at least 92%, at least 95%, at least 98%, at least 99% selectivity.

In some embodiments, the pharmaceutical composition is for use in the prevention or treatment of a disease or disorder associated with abnormal activity of COX and/or Hep within the subject. In some embodiments, the pharmaceutical composition is for use in the prevention or treatment of inflammation and/or pain associated with abnormal activity of COX and/or Hep within the subject. In some embodiments, abnormal activity comprises increased enzymatic activity, wherein increased is by at least 20%, at least 50%, at least 70%, at least 75%, at least 80%, at least 85%, at least 90%, at least 200%, at least 1000%, or more including any range between.

In some embodiments, the pharmaceutical composition/conjugate of the invention is for use in treatment or prevention of a kidney disease in a subject in need thereof. In some embodiments, kidney disease comprises acute kidney disease. In some embodiments, kidney disease comprises acute kidney injury (AKI). In some embodiments, kidney disease comprises chronic kidney disease.

In some embodiments, treatment, prevention, or both comprises reduction of: kidney/body weight %, creatinine levels, blood urea nitrogen levels, or any combination thereof.

In some embodiments, creatinine levels comprises creatinine levels in sample of a subject. In some embodiments, a sample comprises a blood sample and/or urine sample.

In some embodiments, reduced is compared to a control subject. In some embodiments, a control subject comprises a non-treated subject. In some embodiments, a control subject comprises a subject afflicted with kidney disease that is not treated therefor.

In some embodiments, reduction is by at least 5%, 10%, 20%, 30%, 40%, 50%, 60%, 70%, 80%, 90%, 99% or any value and range therebetween. Each possibility represents a separate embodiment of the invention.

In some embodiments, the pharmaceutical composition is for delivery of a conjugate of the invention into a brain of a subject in need thereof. In some embodiments, the pharmaceutical composition is for delivery of NSAID into a brain of a subject in need thereof. Without being limited to any theory or mechanism, it is postulated that trehalose induces or enhances penetration of an NSAID across the blood-brain-barrier (BBB).

In some embodiments, trehalose induces or increases BBB penetration and/or accumulation of an NSAID.

In some embodiments, the pharmaceutical composition is for increasing BBB penetration of an NSAID. In some embodiments, the pharmaceutical composition is for increasing a concentration of an NSAID within a brain of a subject.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

In some embodiments, the pharmaceutical composition comprising the conjugate of the invention is in a unit dosage form. As used herein, the term “unit dosage” means that each unit comprises the conjugate in an amount equivalent to a body weight dose ranging between 0.5 and 5 mg/kg, between 0.5 and 1.5, between 1.2 and 2.6, between 1 and 5, between 1 and 3, between 2 and 4, between 1 and 5, between 1.25 and 3.75 mg/kg body weight, including any value in between.

In some embodiments, the unit dosage form is in the form of a tablet, capsule, lozenge, wafer, patch, ampoule, vial or pre-filled syringe. In some embodiments, the pharmaceutical composition is prepared by any of the methods well known in the art of pharmacy.

In some embodiments, in vitro assays may optionally be employed to help identify optimal dosage ranges. The precise dose to be employed in the formulation depends on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, nasal, ophthalmic, transdermal, subcutaneous, intramuscular, intraperitoneal or intravenous routes of administration. In some embodiments, the route of administration of the pharmaceutical composition will depend on the disease or condition to be treated.

In some embodiments, suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, for oral applications, the pharmaceutical composition is in the form of a tablet or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the method of the invention comprises a preliminary step of selecting a subject suitable for treatment with the conjugate of the invention, wherein the preliminary step is performed prior to the administration step. In some embodiments, the preliminary step comprises determining whether the subject has abnormal (e.g. increased) cyclooxygenase (COX) activity, (e.g. increased) abnormal heparinase (Hep) activity or both, wherein abnormal is as compared to a healthy subject. In some embodiments, the subject suitable for treatment has an abnormal (e.g., enhanced) COX and/or Hep activity, and/or COX and/or Hep expression. In some embodiments, the abnormal activity/expression can be determined by standard detection methods, e.g. by measuring COX and/or Hep expression/activity within a sample (e.g. blood sample, tissue sample, etc.) derived from the subject. In some embodiments, abnormal Hep activity/expression is associated with enhanced lipase and/or amylase levels within a sample derived from the subject. In some embodiments, abnormal Hep activity/expression is determined by determining lipase and/or amylase concentration within a sample derived from the subject, while an enhanced lipase and/or amylase concentration as compared to a healthy subject is indicative of abnormal Hep activity/expression.

In some embodiments, the subject is afflicted with an inflammatory disease. In some embodiments, inflammatory disease comprises brain inflammation. In some embodiments, the subject is afflicted with pain (such as headache, or migraine). In some embodiments, the subject is afflicted with a neurological disorder (such as epilepsy). In some embodiments, the subject is afflicted with seizures.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

According to another aspect, there is provided a method for treating or preventing a COX-related disease, a Hep-related disease, or both, in a subject in need thereof.

According to another aspect, there is provided a method for preventing or treating a kidney disease in a subject in need thereof.

As used herein, the expressions “COX-related” and “Hep-related” disease(s), encompass any disease, or a symptom associated therewith, that is induced by, involves, enhanced by, promoted by, characterized by, propagated by, any equivalent thereof, or any combination thereof, COX activity or Hep activity, respectively, as disclosed herein.

In some embodiments, COX activity comprises oxygenation of arachidonic acid such that a cyclopentane hydroperoxy endoperoxide is produced (also known as prostaglandin G₂); reduction of cyclopentane hydroperoxy endoperoxide such that prostaglandin H₂ is produced, or both.

In some embodiments, COX activity comprises peroxidase activity.

In some embodiments, Hep activity comprises endoglycosidase activity.

In some embodiments, Hep activity comprises cleavage of heparan sulfate. In some embodiments, heparan sulfate comprises polymeric heparan sulfate. In some embodiments, heparan sulfate comprises polymers comprising heparan sulfate molecules.

In some embodiments, the method comprises administering an effective amount of the pharmaceutical composition of the invention to a subject in need thereof. In some embodiments, the method is for inhibiting or reducing COX activity and/or Hep activity in the subject. In some embodiments, the method is for inhibiting or reducing COX activity and/or Hep activity in the brain of a subject. In some embodiments, COX/Hep activity is as described herein.

In some embodiments, the method comprises administering to a subject a therapeutically effective amount of the pharmaceutical composition of the invention.

In some embodiments, the method comprises administering to a subject a therapeutically effective amount of the conjugate of the invention.

In some embodiments, a kidney disease is acute kidney injury (AKI). In some embodiments, AKI is inflammation-induced AKI. In some embodiments, inflammation is a symptom of the AKI.

In some embodiments, treating or preventing comprises reducing: kidney/body weight (% g), creatinine level, blood urea nitrogen level, or any combination thereof.

In some embodiments, creatinine level comprises blood creatinine level, urine creatinine level, or both.

In some embodiments, reducing is compared to a control. In some embodiments, a control comprises a control subject. In some embodiments, a control subject is afflicted with a kidney disease and is not treated with the pharmaceutical composition of the invention, the conjugate of the invention, or both.

In some embodiments, preventing or treating comprises reducing expression level of a marker selected from neutrophil gelatinase-associated lipocalin (NGAL), kidney injury molecule-1 (KIM-1), or both.

In some embodiments, determining expression level of a marker is in a sample obtained or derived from the subject.

In some embodiments, a samples comprises a biological sample.

In some embodiments, a sample comprises a biopsy obtained or derived from a subject. In some embodiments, a sample comprises DNA, RNA, a protein, a plurality thereof, or any combination thereof, being obtained or derived from a sample of a subject. In some embodiments, a sample comprises a kidney, a tissue thereof, a cell thereof, any fraction thereof, or any combination thereof, of a subject.

In some embodiments, the expression level comprises transcript level of a marker, protein level of a marker, or both.

Methods for determining expression, such as of a transcript and/or a protein are common and would be apparent to one of ordinary skill in the art. Non-limiting examples for such methods of expression determination, include, but are not limited to RT-PCR, real-time RT-PCR, next generation sequencing, western blot, dot-blot, densitometry, among others.

In some embodiments, the method further comprises a step comprising determining kidney/body weight (% g), creatinine level, blood urea nitrogen level, expression level of a marker selected from neutrophil gelatinase-associated lipocalin (NGAL), kidney injury molecule-1 (KIM-1), or both, or any combination thereof, of the subject.

In some embodiments, the determining comprises in vitro and/or ex vivo determining.

A person of skill in the art would acknowledge that ex vivo and/or in vitro refers to methods not applied in or on a body of a subject, such as a human subject.

In some embodiments, the effective amount is equivalent to a body weight dose of the conjugate of the invention ranging between 0.5 and 5 mg/kg, between 0.5 and 1.5, between 1.2 and 2.6, between 1 and 5, between 1 and 3, between 2 and 4, between 1 and 5, between 1.25 and 3.75 mg/kg body weight, including any value in between.

In some embodiments, the method is for increasing BBB penetration of an NSAID. In some embodiments, the method is for increasing a concentration of an NSAID within a brain of a subject. In some embodiments, the method is for increasing cellular internalization of an NSAID. In some embodiments, the method is for increasing bioavailability of an NSAID.

In some embodiments, the method is for preventing or treating brain inflammation. In some embodiments, the method is for preventing or treating a disease associated with brain inflammation. In some embodiments, the method is for preventing or treating pain (e.g., headache, migraine etc.). In some embodiments, the method is for preventing or treating fever. In some embodiments, the method is for preventing or treating platelet adhesion (preventing clot formation). In some embodiments, the method further comprises the primary step of determining the suitability of the subject for treatment by the conjugate/pharmaceutical composition of the invention, wherein the preliminary step is as described hereinabove.

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

Various embodiments and aspects of the present invention as delineated hereinabove and as claimed in the claims section below find experimental support in the following examples.

### EXAMPLES

### Materials and Methods

### Animals

Studies utilized wild type (WT) BALB/c mice and heparanase transgenic (Hpa-Tg) mice in which the human heparanase gene is driven by a constitutive β-actin promoter (19) in a BALB/c genetic background. Animals were fed standard mouse chow and tap water ad libitum. All experiments were approved and performed according to the Technion's guidelines of the Committee for the Supervision of Animal Experiments.

### Induction of Acute Pancreatitis

Mice were injected with either cerulein (intraperitoneally, 50 mg/kg, 5 times at 1 hour apart) (Sigma-Aldrich), or saline (0.9% NaCl) (control group). Additional groups of mice were pretreated with either Aspirin (250 mg/kg, SC) and Trehalose (2000 mg/kg, ip) alone or combined. Additional groups of animals were pre- and post-treated with Aspirlose (10 mg/mouse). In the next step, the inventors examined the pancreato-protective effects of indomethacin and Diclofenac (16 and 32 mg/kg ip) alone or combined with Trehalose. Finally, the inventors tested the effectivity of “Indose” and “Diclose” at various concentrations against AP. Mice were sacrificed 24 hours later, and serum samples and pancreatic tissue were collected for measurements of blood amylase and lipase levels, pancreatic index (pancreas/body weight ratio), and for histological analysis. Portion of pancreas tissues were also homogenized, and lysate samples were subjected to immunoblotting.

### Induction of Acute Kidney Injury (AKI)

Renal ischemia was induced by clamping the renal artery of both kidneys for 30 min followed by reperfusion (I/R). The inventors tested the effectivity of “Indose” and “Diclose” before induction of ischemia (e.g., “prophylactic effect” or “pre-treatment”) or after (e.g., “treating effect” or “post-treatment”), using the dosing as disclosed hereinabove. Mice were sacrificed 48 hours later, and serum samples and kidney tissue were collected for measurements of blood creatinine and urea nitrogen levels, kidney index (kidney/body weight ratio), and for histological analysis. Portion of kidney tissues were also homogenized, and lysate samples were subjected to mRNA extraction, and expression analysis of neutrophil gelatinase-associated lipocalin (NGAL) and kidney injury molecule-1 (KIM-1).

### Light Microscopy

Pancreatic tissue samples were fixed in 10% neutral-buffered formalin progressively dehydrated in graduated alcohol and embedded in paraffin. Five-micron (5 μm) sections were stained with hematoxylin and eosin (H&E).

### Electron Microscopy

Tissue samples were stabilized with glutaraldehyde 2% and osmium tetroxide (OsO4) 4%, followed by dehydration in graded ethanol, embedding in commercially available resin media, and staining with uranyl acetate and lead nitrate to achieve better image contrast for further examination in JEOL 1011 transmission electron microscope.

### Statistical Analysis

Data are presented as mean of repeated measurements±standard error (S.E.M). Comparison between two parametric groups was performed using the unpaired Student t test after testing for equality of variances. More than two paired groups were tested using the one-way analysis of variance (ANOVA) test for repeated measurements, followed by the Tukey post-hoc test for multiple comparisons.

### Example 1

The inventors synthesized various NSAID-trehalose conjugates according to the synthetic procedures disclosed hereinbelow.

Aspirin, Diclofenac sodium salt, D+ trehalose dihydrate, Indomethacin, N,N-Dicyclohexylcarbodiimide, 4-Dimethylaminopyridine, Hydroxybenotriazole, Dimethylformamide and silica gel 230-400 mesh were purchased from Sigma Aldrich.

### Asprilose

The inventors studied various trehalose:aspirin ratios in order to obtain the desired mono-, and/or diester in an acceptable yield. Based on these experiments a trehalose:aspirin ratio of 2:1 has been chosen. The synthesis of asprilose has been performed as follows:

Aspirin (acetyl salicylic acid) (1.8 gr (10 mmole)), N,N-Dicyclohexylcarbodiimide (2.06 gr (10 mmole), 4-Dimethylaminopuridine (1.22 gr (10 mmole)), Hydroxybenotriazole (1.35 gr (50 mmole)) and Trehalose (6.84 gr (20 mmole)) were dissolved in Dimethylformamide (20 ml) and stirred for 1-10 hours at room temperature. Precipitated dicyclohexylurea was removed by filtration. Solvent was removed by evaporation (Rotavap). The resulting Asprilose (a mixture of mono and diester) was purified on a reverse phase silica gel C-18 chromatography column, using a gradient of a mobile phase composed of a mixture of water and an organic water miscible solvent (e.g., methanol, ethanol, acetonitrile, ethyl acetate).

Chemical structures of the purified mono and diester was verified by LC-MS and NMR.

### Diclose & Indose

Diclose and Indose have been synthesized according to a similar procedure, as described hereinbelow.

The reaction has been performed in a 100 ml three necked flask equipped with thermometer, magnetic stir, and condenser with Nitrogen bubbler.

Indomethacin (3.57 gr (10 mmole) or 2.95 g Diclofena (10 mmol), has been added to the reaction flask, followed by addition of DCC (2.06 gr (10 mmole), 4-DIMAP (1.22 gr (10 mmole)), HOBT (1.35 gr (10 mmole)) and Trehalose (6.84 gr (20 mmole). Add DMF 25 ml and stirred for 24 hours at room temperature. Precipitated dicyclohexylurea was removed by filtration and washed with 5 ml DMF. The solvent was removed by evaporation (Rotavap) using a high vacuum of 1 mmHg at 45° C.

The oily residue was dissolved in methanol and purified using a Silica gel 230-400 mesh column chromatography. A gradient of methanol 5-25% in ethyl acetate was used as eluent.

Trehalsoe-indomethacin conjugate (Indose) and Trehalose-dichlorofenac conjugate (Diclose) have been obtained as pale-yellow solids, resulting in 32% and 15% yield, respectively.

### Analytical and Structure Analysis

### Trehalose-Indomethacin (Indose)

HRMS: (m/z:): M+Na calculated for C₁₃H₃₆NO₁₄ClNa, 704.1722, Found: 704.1727.

1HNMR (400 MHZ, CD₃OD), delta, ppm 7.71 (dd, J1=8 Hz, J2=4 Hz, 2H Ar), 7.58 (dd, J1=8 Hz, J2=4 Hz, 2H, Ar), 7.01 (dd, J1=4 Hz, 1H Ar), 6.90 (d, J=8 Hz, 1H Ar), 6.67 (dd, J1=8 Hz, J2=4 Hz, 1H, Ar), 5.0 (d, J=4 Hz, 1H), 4.42 (dd, J1=12 Hz, J2=4 Hz, 1H), 4.23 (dd, J1=8 Hz, J2=4 Hz, 1H), 4.0-3.9 (M, 1H), 3.82 (S, 3H), 3.77 (t, J=4 Hz, 1H), 3.76 (S, 2H), 3.72 (dd, J1=8 Hz, J2=4 Hz, 1H), 3.65 (dd, J1=12 Hz, J2-8 Hz, 1H), 3.38 (dd, J1=12 Hz, J2=4 Hz, 1H), 3.27 (dd, J1=8 Hz, J2=4 Hz, 1H), 3.27 (dd, J1=8 Hz, J2=4 Hz, 1H), 3.21 (t, J=8 Hz, 1H), 2.33 (S, 3H).

¹³C NMR: (400 MHZ, CD₃OD), delta ppm: 172.9, 170, 157.7, 140.3, 137.2, 135.8, 132.6, 132.4, 132.2, 130.4, 116.1, 114.3, 112.7, 105.8, 102.9, 95.3, 95.15, 74.8, 74.5, 74, 73.2, 72.0, 71.9, 71.5, 56.3, 30.95, 17.7, 14.6, 13.8, 13.6.

### Trehalose-Dichlorofenac (Diclose)

HRMS: (m/z): calculated for C₂₆H₃₁NO₁₂Cl₂Na, 642.1121, Found 642.1143.

1HNMR (400 MHZ, DMSO-d6), delta, ppm: 7.70-7.76 (m, 4H, Ar), 7.02 (d, J=4 Hz, 1H, Ar), 6.90 (d, J=8 Hz, 1H, Ar), 6.71 (dd, J1=12 Hz, J2=4 Hz, Ar), 5.06 (d, J=8 Hz, 1H), 4.95 (br S 1H), 4.85 (d, J=8 Hz, 1H), 4.86 (t, J=8 Hz, 3H), 4.68 (dd, J1=12 Hz, J2-8 Hz, 2H), 4.35 (t, J=4 Hz, 1H), 4.27 (d, J=12 Hz, 1H), 4.08 (dd, J1=12 Hz, J2=4 Hz, 1H), 3.94-3.91 (m, 1H), 3.66-3.63 (m, 1H), 3.6-3.4 (m, 5H), 3.25-3.15 (m, 4H).

The reaction was followed up by TLC using 25% methanol in ethyl acetate. The esters have an aromatic ring having a strong absorbance in the UV range; thus, it was detected using UV lamp. The trehalose derivative of Diclofenac and Indomethacin was also detected using spray of anisaldehyde-sulfuric acid ethanol. A green spot was obtained at 120° C. on the TLC plate.

### Example 2

Previous studies have highlighted the keen involvement of heparanase in the pathogenesis of inflammatory diseases including acute pancreatitis (AP). Specifically, the inventors evidenced that pancreatic heparanase expression and activity are significantly increased following cerulein-induced AP. Moreover, pancreas edema and inflammation, as well as the induction of cytokines and signaling molecules following cerulein administration were attenuated markedly by PG545 and SST0001, selective heparanase inhibitors, implying that heparanase plays a significant role in AP.

Notably, all the above features appear even more pronounced in transgenic mice over expressing heparanase, suggesting that these mice can be utilized as a sensitive model system to reveal the molecular mechanism by which heparanase functions in AP.

### Heparanase Expression and Activity in the Induced AP

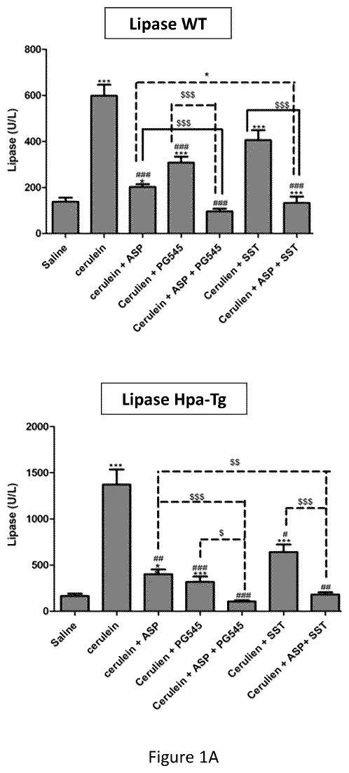

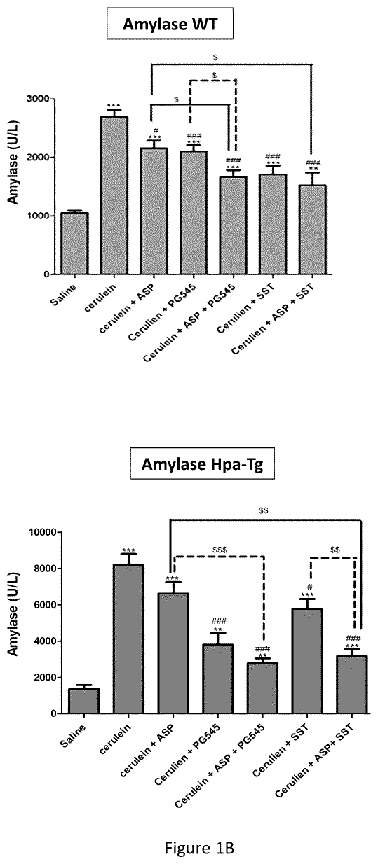

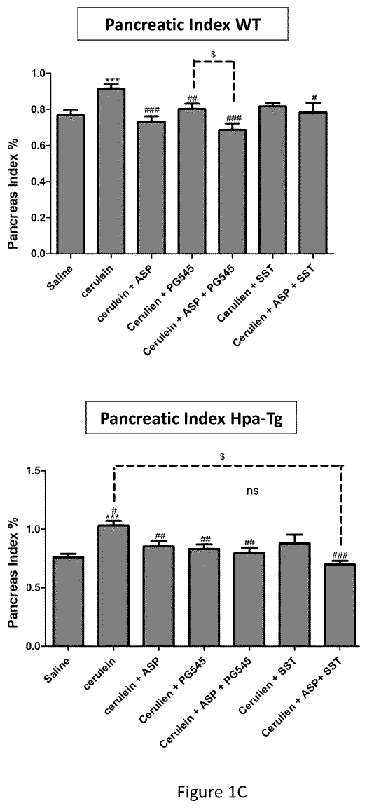

Serum levels of amylase and lipase typical of AP were increased substantially (4-fold) in cerulein-treated WT mice (FIG. 1) and even higher ˜7-fold induction of lipase and amylase levels was noted in Hpa-Tg mice following cerulein treatment (FIG. 1). In addition, AP was associated with pancreatic edema as evident by increased pancreatic index (% Pancreatic weight/Body weight) (FIG. 1). Notably, the elevation in biochemical parameters and pancreatic edema was markedly reduced by the heparanase inhibitors PG545 and Roneparstat in WT and Hpa-Tg mice (FIG. 1), altogether implying that heparanase plays a substantial role in AP.

### Histological Analyses

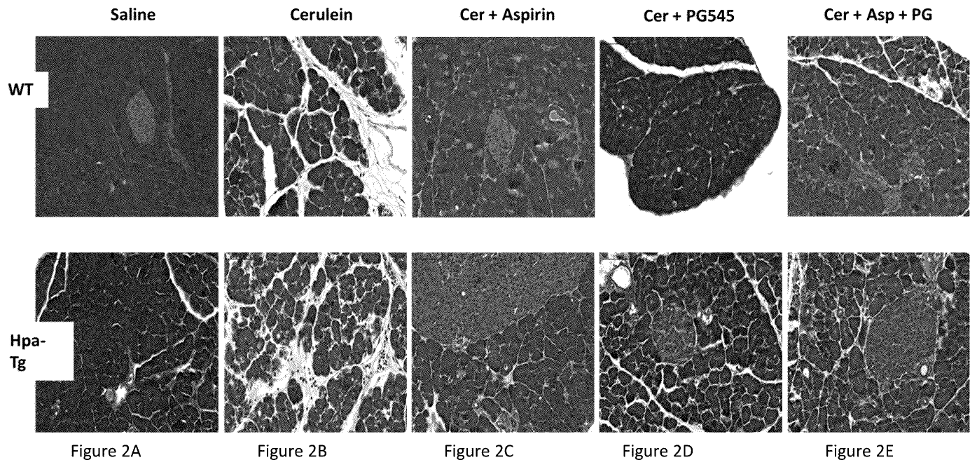

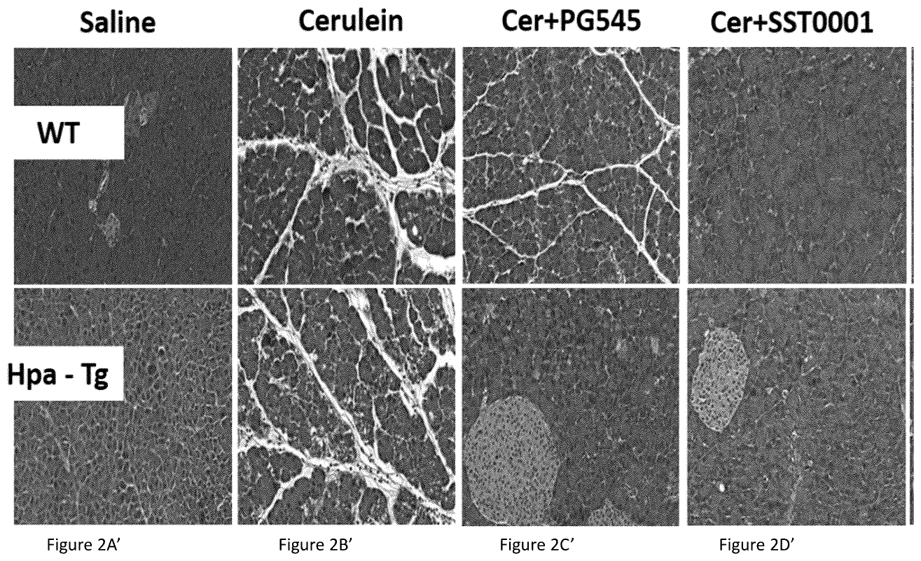

To examine further the involvement of heparanase in the pathogenesis of AP, pancreatic morphology was evaluated in WT and Hpa-Tg mice by histopathological and electron microscopy analyses. Cerulein treatment resulted in typical edema in WT mice and even more sever edema was noted in Hpa-Tg mice exposed to cerulein (FIG. 2). Notably, tissue edema was markedly restored in mice treated with PG545 and SST0001 (FIG. 2), further supporting the notion that heparanase plays a role in AP.

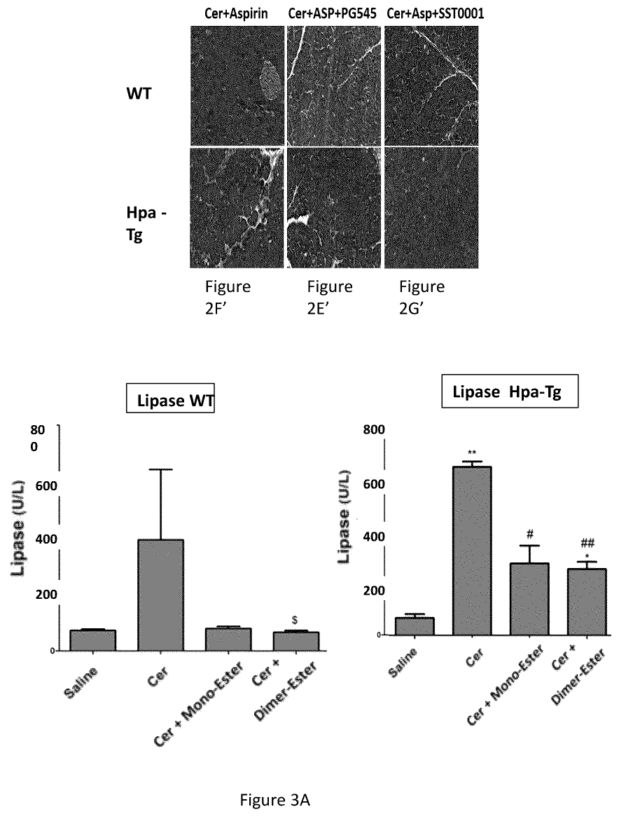

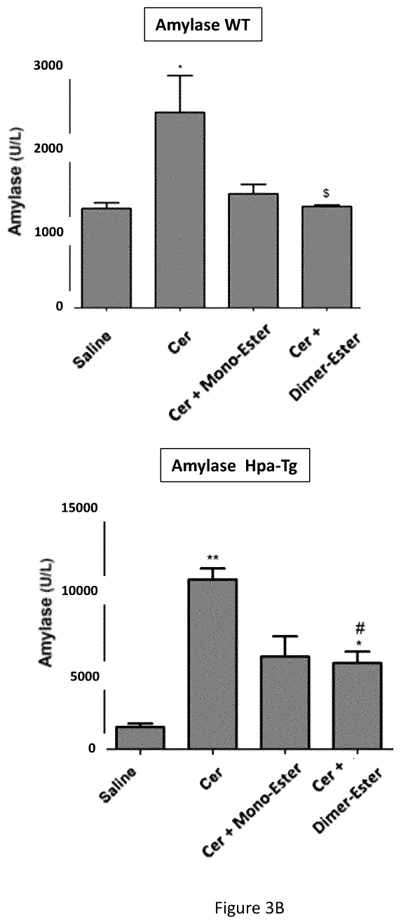

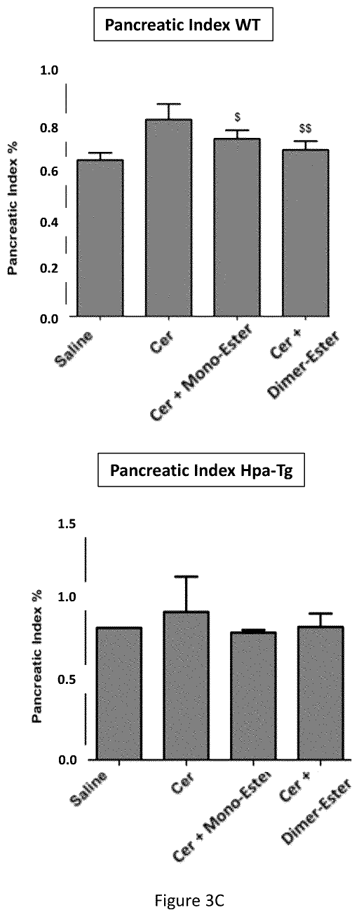

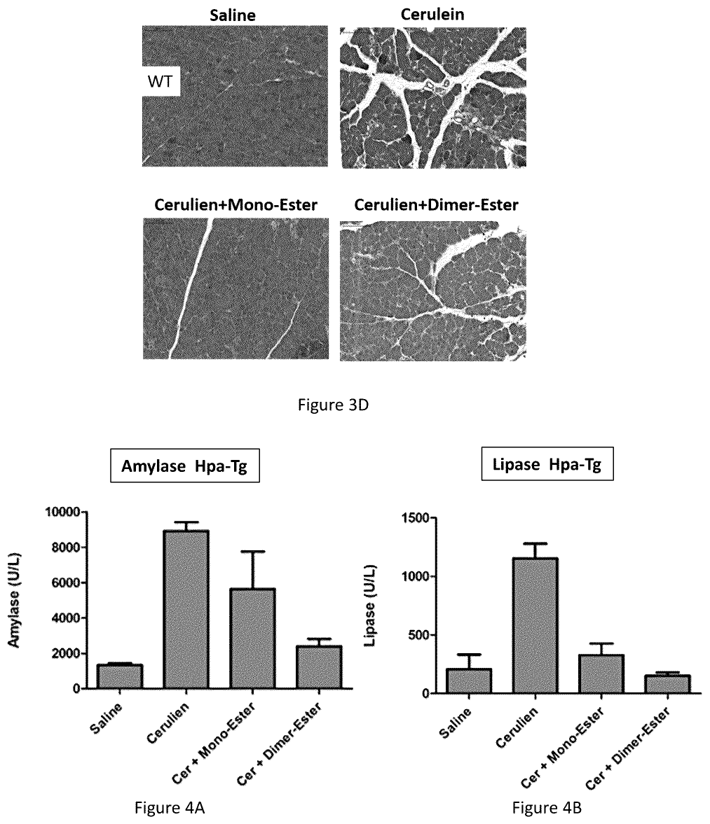

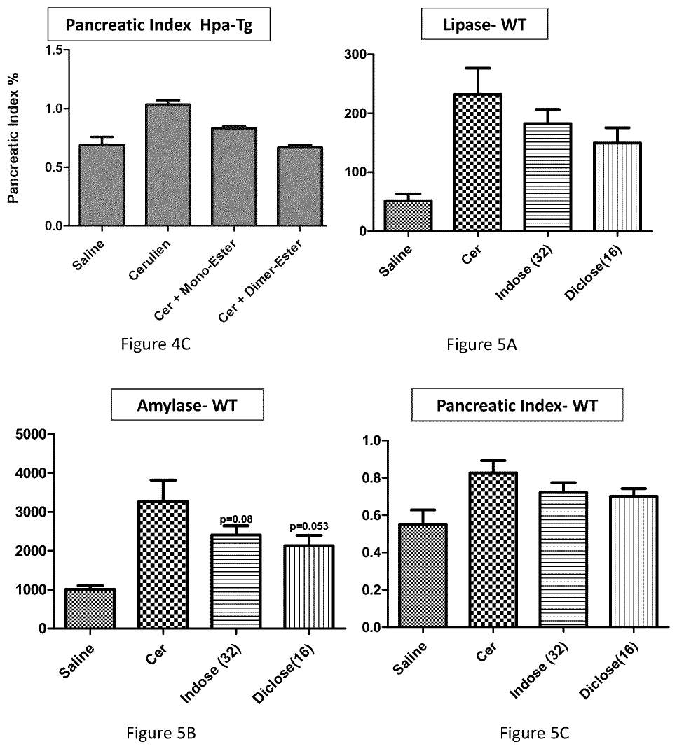

In the next step, the inventors examined the effects of Asprilose as pre and post treatment on AP and the results are presented in FIGS. 3-4. As can be seen, administration of Asprilose (mono or dimer) either as early or post treatment exerts pancreato-protective effects against AP. The beneficial effects of Aspirlose was evident at the histological levels too (FIG. 3). It is postulated that this new compound may be efficient as a novel therapeutic agent for the treatment of AP.

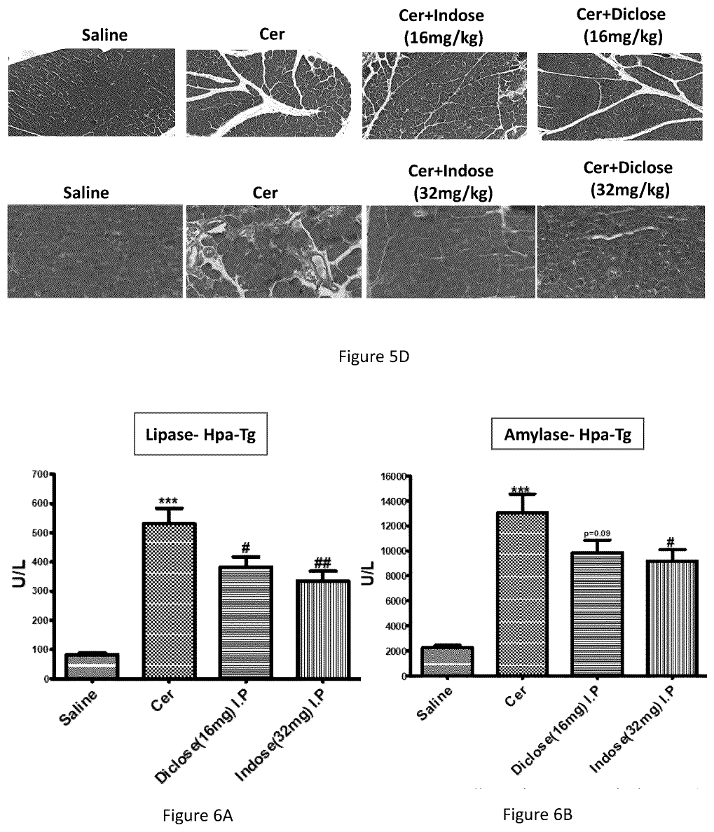

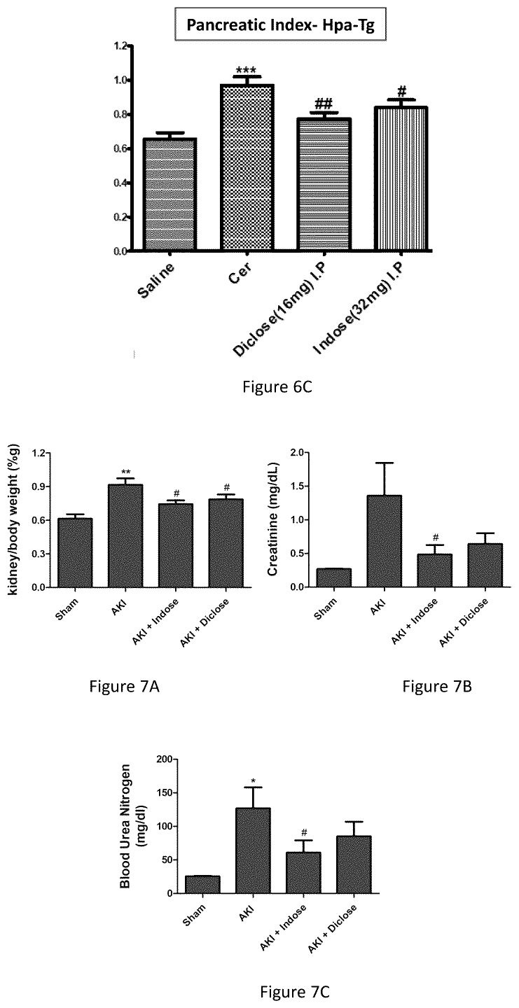

Moreover, the inventors examined the pancreato-protective effects of Indose and Diclose in AP. The newly synthesized compounds, “Indose” or “Diclose”, were well tolerated and effective against AP in WT and Hpa-Tg mice and reduced the severity of AP at 16 and 32 mg/kg doses (see FIGS. 5 and 6, respectively).

Further, the inventors examined the acute toxicity of the compounds Indose and Diclose. A series of dosages: 8 mg/kg, 32 mg/kg, 64 mg/kg, 128 mg/kg, 256 mg/kg, 512 mg/kg, 768 mg/kg, 1024 mg/kg, 1,100 mg/kg, and 1,200 mg/kg were injected by intraperitoneal injection in the presence of sterile water as carrier. Each dosage was injected to a group of 5 wild type mice. Indose exhibit a LD50 of 900 mg/kg and Diclose a LD50 of 780 mg/kg. No mortality was observed, for both Indose and Diclose, at a dosage of 500 mg/kg, indicating both compounds have low toxicity.

To this end, the new pharmaceutical compositions of the invention have a negligible toxicity and showed a significant efficacy in-vivo. Thus, it is postulated that the pharmaceutical compositions or the compounds of the invention may provide a novel therapeutic maneuver for the treatment of disease(s)/condition(s) disclosed herein.

### Example 3

The inventors used the ischemia-reperfusion (I/R)-AKI model in mice. The pathogenesis of this disease was evaluated based on kidney edema, blood creatinine (Scr), and blood urea nitrogen (BUN) levels were determined 48 hours following AKI induction. Further, the inventors performed histopathological analysis, as well as expression analysis of the renal injury biomarkers neutrophil gelatinase-associated lipocalin (NGAL), kidney injury molecule-1 (KIM-1).

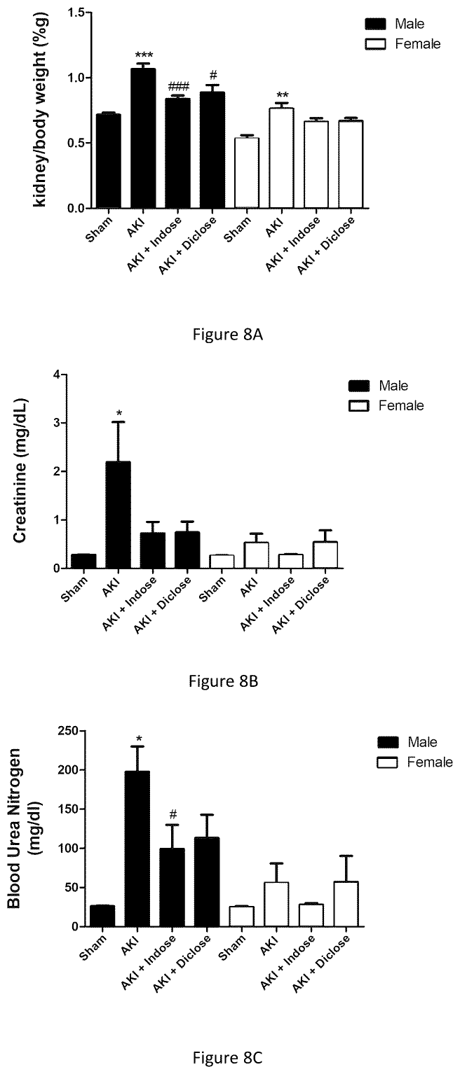

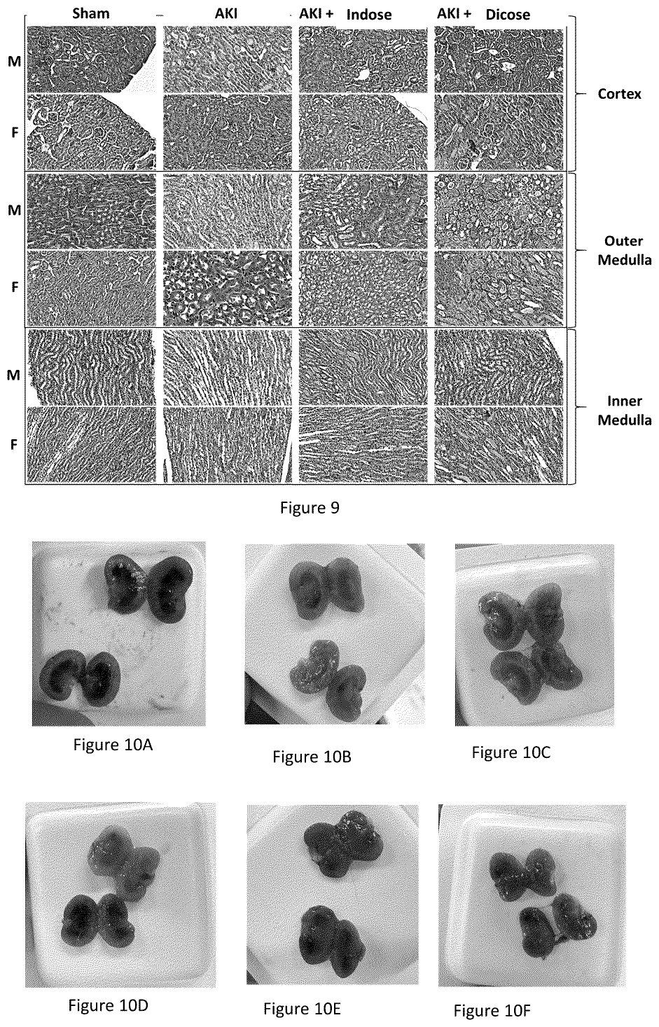

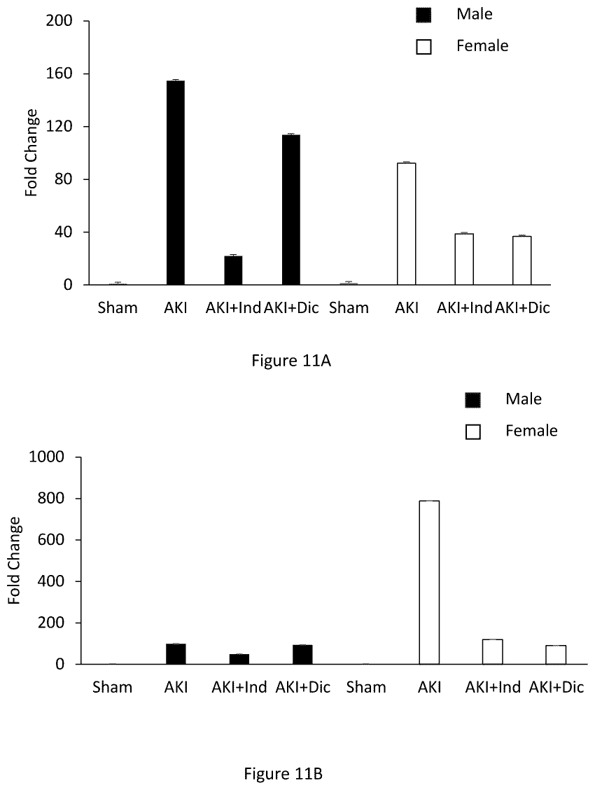

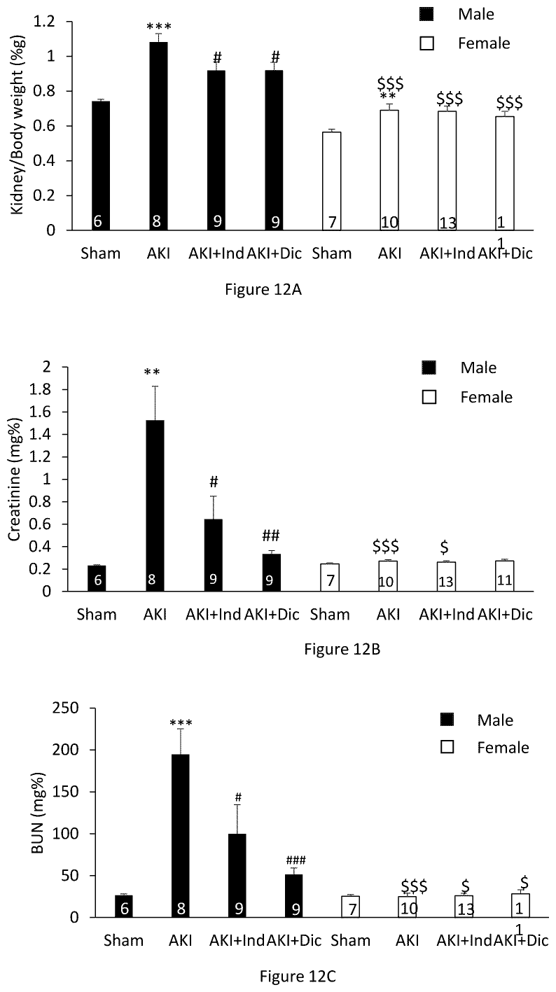

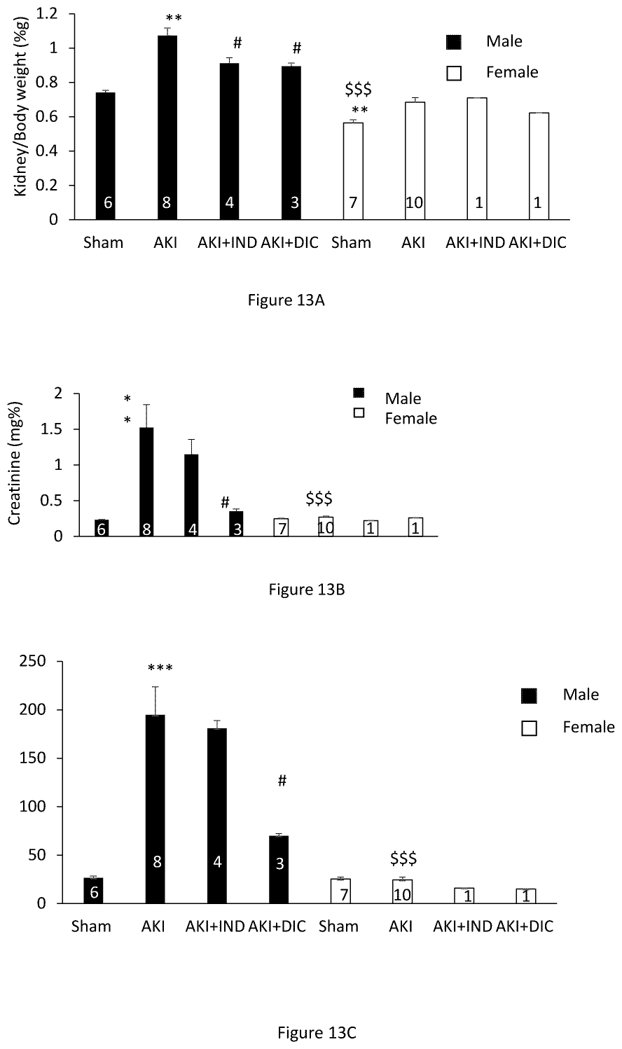

AKI was induced in the absence or presence of pretreatment with Indose (16 mg/kg, i.p.) or Diclose (32 mg/kg, i.p.), respectively in Hpa-Tg mice (males and females). Untreated Hpa-Tg male and female mice that underwent sham operation served as control group. The gross morphology (FIG. 10) and histopathology (FIG. 9) of kidneys of mice pretreated with either Diclose or Indose resembled kidneys of the ‘Sham’ control group. As expected, kidney weight (KW) normalized to body weight (BW) was significantly increased following AKI in untreated males and females, due to edema and inflammatory response (FIGS. 7A and 8A). When the animals were pretreated with Indose or Diclose KW/BW was significantly reduced. Measurement of serum creatinine (SCr) and blood urea nitrogen (BUN) after 48 h from AKI induction, revealed significant elevation of these two renal dysfunction biomarkers following AKI in male untreated mice (FIGS. 7B-7C and 8B-8C). Pretreatment with Indose and Diclose remarkably attenuated this increase, suggesting renoprotection of the kidney function. Both Diclose and Indose have effectively reduced the expression level of NGAL and KIM-1 in female mice (FIGS. 11A-11B). In male mice pretreated with Diclose and Indose, a reduction in NGAL expression was also observed (FIG. 11A). This trend persisted in female mice although the elevation in SCr and BUN following AKI was to a lesser extent. Similar results were obtained in WT FVB/N strain, which is known to be sensitive to AKI (FIGS. 12A-12C). Noteworthy, the inventors performed additional set of experiments in this strain, where Indose or Diclose were administered short time after the induction of AKI (‘Post-treatment’) and showed that both Diclose and Indose provide curative effects of AKI, with the effects of Diclose being more pronounced (FIGS. 13A-13C).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A pharmaceutical composition, comprising a therapeutically effective amount of a conjugate and a pharmaceutically acceptable carrier, wherein the conjugate comprises a di-sugar covalently bound to a Non-Steroidal Anti-inflammatory Drug (NSAID).

2. The pharmaceutical composition of claim 1, wherein said covalently bound is via an ester bond between a carboxy group of the NSAID and a hydroxy group of the di-sugar.

3. The pharmaceutical composition of claim 1, wherein the NSAID comprises one or more compounds selected from the group consisting of: aspirin, diclofenac, indomethacin, naproxen, diflunisal, salsalate, ibuprofen, mefenamic acid, meclofenamic acid, clonixin, licofelone, a COX-2 inhibitor including any pharmaceutically acceptable salt, pharmaceutically active derivative, and a combination thereof.

4. The pharmaceutical composition claim 1, wherein said di-sugar is trehalose.

5. The pharmaceutical composition of claim 1, wherein said NSAID is selected from selected from the group consisting of: aspirin, diclofenac, indomethacin, and any combination thereof.

6. A conjugate, comprising trehalose covalently bound to a Non-Steroidal Anti-inflammatory Drug (NSAID) selected from the group comprising diclofenac, naproxen, diflunisal, salsalate, ibuprofen, indomethacin, mefenamic acid, meclofenamic acid, clonixin, licofelone or a combination thereof.

7. The conjugate of claim 6, wherein said trehalose is covalently bound to a carboxy group of said NSAID.

8. The conjugate of claim 7, wherein said covalently bound is via a bond selected from an ester, an amide, a thioester, a carbamate, a carbonate ester, a carbamide, a thiocarbamate, a phosphonate, a phosphodiester, a sulfonate ester, or any combination thereof.

9. The conjugate of claim 6, wherein said trehalose is covalently bound via a hydroxy group at position 2, at position 6 or both.

10. The conjugate of claim 6, represented by Formula 1: 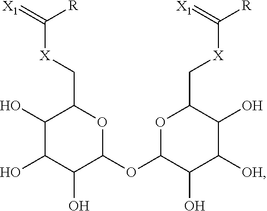
or of Formula 1a: 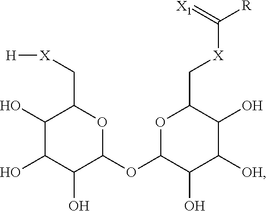
wherein each R independently comprises decarboxylated diclofenac, decarboxylated indomethacin, decarboxylated naproxen, decarboxylated diflunisal, decarboxylated salsalate, decarboxylated paracetamol, decarboxylated ibuprofen, decarboxylated mefenamic acid, decarboxylated meclofenamic acid, decarboxylated clonixin, or decarboxylated licofelone, and each X or X₁ independently comprises O, S, or NH.

11. The conjugate of claim 6, comprising any of:

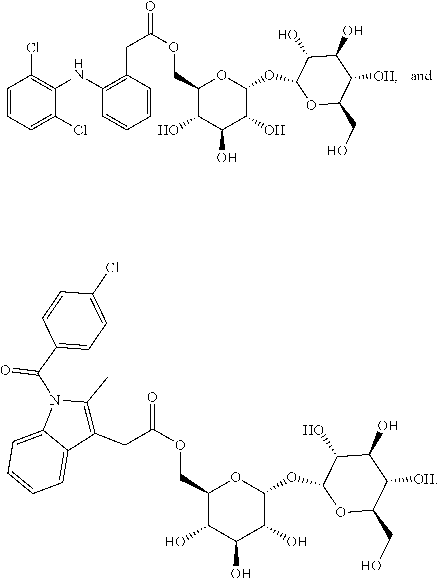 [SMILES: O=C(Cc1ccccc1Nc1c(Cl)cccc1Cl)OC[C@H]1O[C@H](O[C@H]2O[C@H](CO)[C@@H](O)[C@H](O)[C@H]2O)[C@H](O)[C@@H](O)[C@@H]1O ; Cc1c(CC(=O)OC[C@H]2O[C@H](O[C@H]3O[C@H](CO)[C@@H](O)[C@H](O)[C@H]3O)[C@H](O)[C@@H](O)[C@@H]2O)c2ccccc2n1C(=O)c1ccc(Cl)cc1]

12. A pharmaceutical composition comprising the conjugate of claim 6, and a pharmaceutically acceptable carrier.

13. A method for preventing or treating a COX-related disease, a Hep-related disease, or both, in a subject in need thereof, the method comprising administering to said subject a therapeutically effective amount of the pharmaceutical composition of claim 1, thereby, treating a COX-related disease, a Hep-related disease, or both, in the subject.

14. A method for preventing or treating a kidney disease in a subject in need thereof, the method comprising administering to said subject a therapeutically effective amount of the pharmaceutical composition of any one of claim 1, thereby, treating a kidney disease in the subject.

15. The method of claim 14, wherein said kidney disease is acute kidney injury (AKI).

16. The method of claim 14, wherein said treating or preventing comprises reducing any one of: kidney/body weight (% g), creatinine level, blood urea nitrogen level, and any combination thereof, in said subject compared to a control subject, and optionally wherein said creatinine level is blood creatinine level, urine creatinine level, or both.

17. The method of claim 14, wherein said preventing or treating comprises reducing expression level of a marker selected from neutrophil gelatinase-associated lipocalin (NGAL), kidney injury molecule-1 (KIM-1), or both, in a kidney of said subject, compared to a control subject.

18. The method of claim 17, wherein said expression level comprises transcript level of said marker, protein level of said marker, or both.

19. The method of claim 18, wherein said control subject is afflicted with said kidney disease and not being treated with any one of said pharmaceutical composition and said conjugate.

20. A method for inhibiting or reducing enzymatic activity within a subject in need thereof, comprising administering the pharmaceutical composition of claim 1 to said subject, wherein the enzymatic activity comprises COX activity, Hep activity, or both, optionally wherein said reducing enzymatic activity comprises preventing or treating any condition selected from the group consisting of: inflammation, fever, and pain, in said subject, and optionally wherein said inflammation comprises pancreatitis.
